(19) United States
(12) Patent Application Publication
(10) Pub. No.: US 2008/0275368 A1
(43) Pub. Date: Nov. 6, 2008

(54) Apparatus For Detecting Axial Force In The Digestive System

(75) Inventors: Hans Gregersen, Hornslet (DK); Lene Kehlet Drud, Hornslet (DK)

Correspondence Address: FOLEY AND LARDNER LLP;SUITE 500, 3000 K STREET NW, WASHINGTON, DC, 20007, US

(73) Assignee: Ditens A/S

(21) Appl. No.: 10/567,120

(22) PCT Filed: Aug. 4, 2004
(86) PCT No.: PCT/DK04/00522

§ 371 Date: Sep. 28, 2007

(30) Foreign Application Priority Data
Aug. 4, 2003 (DK) PA 2003 01126

(51) Int. Cl. A61B 5/00

(52) U.S. Cl. 600/593

(57) ABSTRACT

An apparatus and a method for measuring deformations and force applied to a probe are disclosed. The system may be a mechanical system a physical system or a biological system such as e.g. a bodily hollow system. The apparatus comprises an elongated elastic probe, a conducting medium attached to or contained by the probe, two or more electrodes being electrically connected by the conducting medium, the electrodes being attached to the probe, and the apparatus furthermore comprising means for measuring an electrical parameter, such as the potential difference between at least two of the number of electrodes, the measured electrical parameter being indicative of a deformation of the probe in at least the longitudinal direction of the elongated probe. The force applied to the probe may be determined from a pre-calibration of the electrical parameter-force relationship of the probe.

### FIELD OF THE INVENTION

The invention relates to an apparatus and a method for measuring deformations and force of a system. The system may be a mechanical system, a physical system or a biological system such as e.g. a bodily hollow system. Finally, the invention relates to uses of the apparatus according to the invention.

### BACKGROUND OF THE INVENTION

The function of visceral organs like the gastrointestinal tract, the urinary tract and the blood vessels is to a large degree mechanical. The following introduction refers mainly to the gastrointestinal tract but the invention relates to similar applications in other hollow organs and even to measurement of deformation and forces inside tissues such as in muscle, in plants and in engineered structures.

In the gastrointestinal tract, contents received from the stomach are propelled further down the intestine and mixed with secreted fluids to digest and absorb the food constituents. The biomechanical properties of the small intestine in vivo are largely unknown, despite the fact that the distensibility is important for normal function, and altered mechanical properties are associated with gastrointestinal (GI) diseases. Data in the literature pertaining to the mechanical aspects of GI function are concerned with the contraction patterns, the length-tension relationship in circular and longitudinal tissue strips in vitro, flow patterns, the compliance and the tension-strain relationship in vivo. The methods traditionally used for clinical or basic investigations of the small intestine are endoscopy, manometry and radiographic examinations. Although these methods provide important data on the motor function, little attention has been paid to biomechanical parameters such as wall tension and strain and the relation between biomechanical properties and sensation. During the past two decades, impedance planimetry was used in gastroenterology to determine wall tension and strain in animal experiments and human studies. Impedance planimetry provides a measure of balloon cross-sectional area and is therefore a better basis than volume measurements for determination of mechanical parameters such as tension and strain in cylindrical organs. Impedance planimetry, however, only provides a measure of circumferential tension and as such no measurement of axial forces (such as traction force during swallowing or peristalsis) is provided by impedance planimetry. The same accounts for manometry that provides a measurement of pressure but no axial force. A few scientific papers have described the use of a force transducer in terms of a strain gauge mounted on a probe inserted into the lumen of the gastrointestinal tract. The purpose was to measure the axial force during contractions (swallows in the esophagus). The strain gauge technique suffers from high expenses, signal drift and difficulties mounting the strain gauges on the probe.

It is well known that distension of the gastrointestinal tract elicits reflex-mediated inhibition and stimulation of motility via intrinsic or extrinsic neural circuits and induces visceral perception such as pain. Previous studies demonstrated that mechanoreceptors located in the intestinal wall play an important role in the stimulus-response function. The receptors are stimulated by mechanical forces and deformations acting in the intestinal wall due to changes in the transmural pressure. Thus, the mechanical distension stimulus and the biomechanical tissue properties must be taken into account in studies of the sensory-motor function in the intestine: It is likely that symptoms and pain are associated with forces and deformation in axial direction in a hollow organ. This puts emphasis on developing a reliable and inexpensive method to measure such properties under a range of functional states of the organ together with other measurements. By functional states means consideration of the muscle physiological state, pharmacological relaxation and stimulation of the muscle in the organ, diagnostic procedures, intervention and disease.

There is a considerable interest in improved diagnostics of motor disease of visceral organs. In particular this relates to the esophagus and diseases affecting the esophagus such as gastroesophageal reflux disease systemic sclerosis, spasms and non-cardiac chest pain. A proper test will also be relevant for use in the distal part of the stomach and the intestines in patients with dyspepsia, gastroparesis due to diabetes mellitus and irritable bowel syndrome. The groups of patients with these diseases are huge, for example 10-20 percent of the population suffer from the irritable bowel syndrome.

Mechanical properties have been studied in vitro in muscle tissue strips from various organs. The strips are mounted in a small organ bath between hooks. The strip can be elongated in a controlled way and the resultant force measured. This test is often done in the direction of the longitudinal axis of the muscle fibres. The strips studies have rendered possible studies of isometric and isotonic muscle length-tension diagrams in vitro. Usually the tissue has been studied when influenced by drugs such as muscle relaxants and muscle stimulants, in order to study active and passive tissue properties. The passive curve is normally described as exponential whereas the active curve is bell-shaped, i.e. with a maximum force at a certain strain level. The maximum active tension is presumably reached at a level of optimum overlap between the sliding filaments in the intestinal muscle cells. In vivo no such method exists for studying the properties in the axial direction of a hollow organ like the gastrointestinal tract.

WO 03/020124 describes a method and an apparatus for stimulating and/or measuring visceral pain in a bodily hollow system of a human being or an animal. The method and apparatus is especially well suited for multi-modal stimulation and measuring, where different stimulus modalities are integrated into one stimulus device. The stimuli may be any one or more of the stimuli: mechanical stimulus, thermal stimulus, chemical stimulus and electric stimulus. The stimuli may activate superficial and deeper layers of the hollow system. Distinct responses to the individual stimuli and robust stimulus-response relations are obtained and result in the possibility of comparative studies of different visceral sensations. WO 03/020124 does not specify a specific solution for measurement of axial forces without and with combination with the multi-modal stimulations and measurements.

U.S. Pat. No. 5,617,876 describes a method for measurement of micromotions of the wall of hollow organs. The apparatus consist of a catheter with at least four electrodes affixed to an inner surface of the balloon so when the wall of the balloon is pressed against the organ wall, the electrodes will move and thus record movement of the organ. In the disclosed invention the balloon is the actual recording site and forces are not measured.

US 2003/004434 describes a method for mounting balloons on a catheter using a carrier to slide the balloon over the catheter. This may include catheters with electrodes for impedance planimetry. Whereas the disclosed invention describes a novel method for balloon mounting, it does not in itself provide a measurement system.

U.S. Pat. No. 4,561,450 describes a catheter with electrodes as part of a Wheatstone bridge circuit. The three spaced electrodes are fixed at the inside of a fluid-filled channel in the catheter. The electrical imbalance in the bridge circuit is indicative of pressure changes in the organ. The invention is entirely depending on a Wheatstone bridge solution using a centrally disposed electrode surrounded by two other electrodes in a softer part of the catheter.

### SUMMARY OF THE INVENTION

The object of the present invention may be to record deformations and force of a system in a manner ensuring reliable measuring and to provide an apparatus eliminating or at least to a large extend reducing the number of and/or the magnitude of the disadvantages of the prior art.

This object and other objects may be obtained in a first aspect by an apparatus for measuring the deformation of a system, the apparatus comprising:
an elongated elastic probe,
a conducting medium attached to or contained by the probe, and
a two or more electrodes being electrically connected by the conducting medium, the electrodes being attached to the probe,
   wherein the apparatus furthermore comprising means for measuring an electrical parameter between at least two of the number of electrodes, the measured electrical parameter being indicative of a deformation of the probe in at least the longitudinal direction of the elongated probe.

Further, according to a second object, the above-standing and other objects may be obtained by a method for measuring a deformation of a system by introducing into the system an elongate elastic probe, the probe comprising:
a conducting medium attached to or contained by the probe, and
a two or more electrodes being electrically connected by the conducting medium, the electrodes being attached to the probe,
   wherein a deformation being indicative of a deformation of the probe in at least the longitudinal direction of the elongated probe is measured by measuring an electrical parameter between at least two of the two or more electrodes.

The system may be a mechanical system, a physical system or a biological system such as e.g. a bodily hollow system or a muscle, the system may also be a plant, such as a hollow part of a plant, or an engineered structure. The elongated elastic probe may be such as catheter or a catheter shaped probe made in a material suitable for insertion e.g. into the human or animal body. The probe may e.g. be made of a bio-compatible plastic or polymer material.

The proposed invention is based on measurement of electrical parameter which is easy to do and inexpensive. It is to be understood that the invention deals with the measuring of quantities such as a potential difference, an electrical current and/or an impedance (or resistance), such as quantities related by Ohm's law, thus the invention deals with measurements of electrical properties of a medium. The electrical parameter measurement in a conducting medium may correlate to the deformation of the probe, such as a stretching or contraction of the probe. The deformation of the probe may be the result of an external force applied to the probe, and therefore electrical parameter measurements indicative of a deformation of the probe may yield information of a force applied to the probe, and thus be force measurements.

The apparatus according to the present invention may comprise a number of electrodes, such as four or more electrodes, wherein at least two of the four or more electrodes are measuring electrodes comprising means for measuring the electrical potential between them, and wherein other at least two of the four or more electrodes are generating electrodes comprising means for generating an AC-field between the measuring electrodes. It may be an advantage to provide an apparatus including means for generating an AC-field, since more stable and/or more sensitive measurements may be obtained thereby.

The apparatus according to the present invention may further comprise timer means for determining a timing of a change of the measured electrical parameter. The timer means may be a part of external equipment controlling or handling the measurements

The apparatus according to the present invention may further comprise at least one inflatable balloon or bag situated between a proximal end and a distal end of the probe, and the apparatus comprising means for passing an inflating fluid, preferably a liquid, from the proximal end to the balloon, and where the apparatus optionally is provided with means for measuring at least one physical properties of the balloon.

The measuring of the electrical parameter between at least two of the two or more electrodes may be obtained in correlation with a pressure change inside the balloon, a volume change of the balloon, a determination of the cross-sectional area of the balloon or other changes of the balloon, so as to obtain a correlation between a deformation of the probe and a quantity of the balloon. It may be an advantage to combine balloon distension and measurements of e.g. the axial force, in order to provide a force-tension relationship.

Furthermore, a measurement during thermal stimulus may be performed, where the probe and/or the balloon is filled with a fluid, preferably a liquid, the liquid introducing a change in temperature of the probe and/or balloon, the surface abutting the inner wall of the system is thereby exposed to a thermal stimulus, the deformation of the system may thereby be measured in correlation with the temperature of the fluid inside the probe and/or balloon.

Even further, a measurement during chemical stimulus may be performed, when passing of a chemical substance through a number of the canals inside the probe to a number of openings in side-walls of the probe and out into the hollow system, and where the extension or the contraction of the hollow system is measured in correlation with the composition of the chemical substance.

The chemical substance may be a substance commonly present in the bodily hollow system being measured, such as an acid like HCl in the stomach, or such as bile salts in the gall bladder, or such as water with NaCl in the esophagus. The chemical substance may also be a pharmaceutical substance intended for treatment of diseases in the bodily system being measured, such as smooth muscles relaxants. The chemical substance may even be a substance having special technical or physical properties such as a contrast fluid intended for co-operation with an exterior measuring means such as an X-ray apparatus.

The measurement may be performed in order to determine the passage of the chemical substance past a part of the probe abutting the internal wall of the system, the passage being indicative of the ability of the system to exercise a restraining influence, alternatively to exercise a passing influence, on liquids and solids.

The measurement may be performed during an electrical stimulus, when passing an electrical current through a number of wires in a number of the canals inside the probe, and when passing the electrical current to an outer surface of the probe, the outer surface being a surface abutting the inner wall of the hollow system, and where the extension or the contraction of the hollow system is measured in correlation with the magnitude of the electrical current applied.

The electrical current may be applied during a certain interval of time, and where the extension or the contraction of the hollow system is measured in correlation with the magnitude of the time interval, when the electrical current is applied. Alternative, the electrical current may be applied at a certain frequency of time, and where the longitudinal extension of the hollow system is measured in correlation with the frequency of time, at which the electrical current is applied.

The apparatus may be used for performing measurement anywhere in one of the following bodily systems: the tissue including epitheliuous tissue, connective tissue, skin, and adipose tissue, the skin, the motoric system including the muscles and the bones, or may be used anywhere in one of the following bodily hollow systems: the digestive system including the gastrointestinal tract and the stomach, the urogenital tract including the bladder, the cardiovascular system including the heart, the lymph system, the ear canal including the eustachian canal and the posterior nares.

The apparatus may be used when a bodily hollow system of a person or an animal is being subjected to a number of artificially applied stimuli, the stimuli being any of the stimuli: mechanical stimulus, thermal stimulus, chemical stimulus and electric stimulus.

The apparatus may also be used for performing measurement in non-human and non-animal systems such as in plants and in engineered structures.

### BRIEF DESCRIPTION OF THE FIGURES

Preferred embodiments of the invention will now be described in details with reference to the drawings in which:

FIG. 1 is a schematic view of an apparatus according to the invention and comprising a probe with an electrolyte and two electrodes in a non-stretched state, and the two electrodes in a stretched state,

FIG. 2 is a diagram showing a possible relationship between an electrical potential difference between the electrodes compared to a mass applied to the probe,

FIG. 3 illustrates different embodiments of the probe,

FIG. 4 illustrates different embodiments of electrode configuration, and

FIG. 5 illustrates a probe according to the present invention introduced into the esophagus.

### DETAILED DESCRIPTION OF THE INVENTION

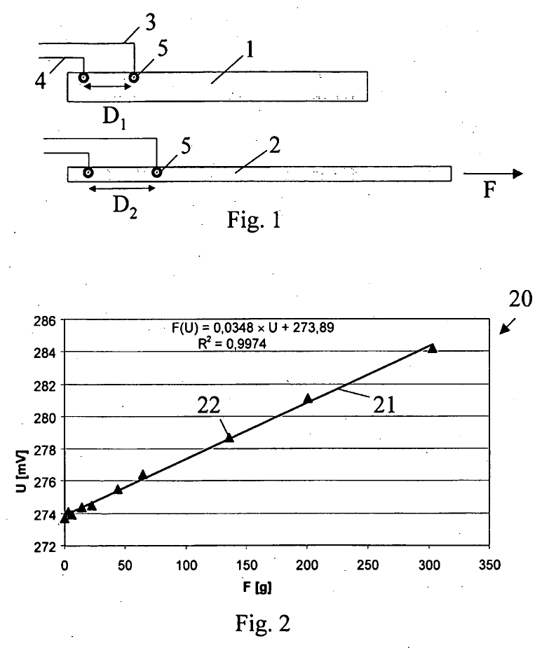

FIG. 1 illustrates one aspect of the measuring principle of the present invention by showing schematically an elongated probe having side walls and exhibiting a hollow inner chamber or channel 1,2. The hollow chamber is filled with a conducting medium, preferably a liquid electrolyte such as water with a solution of NaCl. Two electrodes 5 are provided in the probe. The electrodes are connected to exterior equipment by means of wires 3,4. A distance D₁ is present between the two electrodes for the probe in a relaxed or unstretched condition, whereas the distance is altered to D₂ for the probe in a condition where it is stretched by means of applying a force F in an axial direction. The electrodes are used for measurement of the electrical properties in the fluid between them. When the probe is stretched in axial direction, the electrical impedance will increase due to the longer distance between the electrodes and the smaller diameter in the fluid-filled channel.

By applying Ohm's Law, U=Z×I, where U is the potential difference between the electrodes trough the conducting medium, Z is the electrical impedance of the conducting medium between the electrodes, and I is the current running between the electrodes. A change in the electrical potential difference between two electrodes can occur during the stretching of the probe.

The electrical impedance Z of the conducting medium is determined as Z=ρ×(D/A), where ρ is the electrical resistivity of the conducting medium, D is the distance through the conducting medium between the electrodes at the time of measurement, and A is the cross-sectional area of the conducting medium, also at the time of measurement. Thus, keeping the current constant, the potential difference will vary as function of the distance and cross-sectional area, whereas if the potential difference is kept constant the current will vary.

By applying the physical properties of the probe even further, the change in distance between the electrodes in the non-stretched state compared to the stretched state is a direct result of the force being applied to the probe, since either the distance between the electrodes or the cross-sectional area of the probe, or both, are changed upon the deformation. Thus, a certain change in distance between the electrodes corresponds to a certain force being applied to the probe.

Depending on the choice of material, which the probe is made of, and depending on whether stretching of the probe is maintained within an elastic and no plastic deformation of the probe during stretching, a linear or non-linear relationship between the force being applied and the change of distance between the electrodes may be obtained. A calibration may thus be provided in order to obtain any relationship between the force being applied and the change of distance between the electrodes, and thereby providing a force measurement as a function of the potential difference.

FIG. 2 is a diagram 20 showing the relationship between a force being applied to the probe in longitudinal direction and the electrical potential between the electrodes. The force is expressed as a mass, which the probe is subjected to, said mass in the embodiment of the test being influenced by gravity only. The diagram shows both measuring points 22 as well as a fitted straight line 21. The force is applied to a probe made of PVC, having a diameter of 4.5 mm, being a multi-lumen probe, and the distance between the electrodes being 10 mm in the non-stretched state. The conducting medium is a 0.9% solution of NaCl in water. In this experiment it is evident that the voltage difference U is directly proportional to the force F imposed in longitudinal direction to the probe (in the current experiment done by hanging weights in one end of the vertical oriented probe). However, in other experiments the calibration curve may be non-linear but this can easily be accounted for. Diagrams like the one presented in FIG. 2 may be provided for each probe geometry and material choice and incorporated into control or handling equipment, such as electronic equipment handling the measured data.

Also, if the distance between the electrodes is monitored continuously together with continuously monitoring the time during which the change of distance takes place, a velocity and/or an acceleration of the deformation of the probe between the electrodes can be determined.

It may be important to provide also information about the acceleration and/or velocity of the deformation as a complement to a force measurement, since the force measurement may provide information about the strength of the object inducing the force, but the acceleration and/or velocity of the deformation may provide information about the intensity of the force reaction and the reaction time.

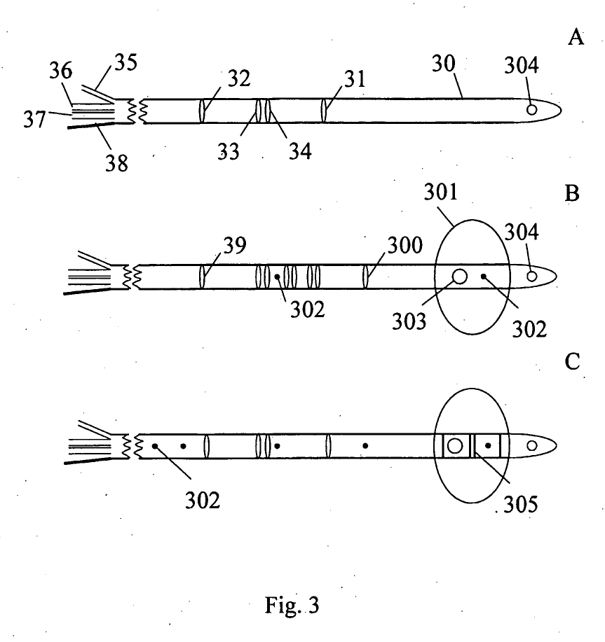

In FIG. 3 examples of embodiments of the probe 30 are illustrated. The probe may be formed as a single or multi-lumen catheter. The number of electrodes may be more than two. In fact improved measurements may be obtained by a four-electrode system with two outer electrodes 31, 32 generating a alternating current of constant magnitude between them and with two electrodes placed between the other electrodes for measurement of the potential difference between them 33, 34. The channel of the probe may be filled with a conducting liquid through an inlet hole 35. The probe may also be provided with an outlet hole, here illustrated as being present in the distal end of the probe, however the outlet may be present anywhere in the channel to establish a fluid perfusion through the channel of probe. It may be an advantage to perfuse fluid in order to avoid air bubbles inside the channel. The probe may be provided with a number of inlet holes 35-37, as well as exterior accessible wires or connectors 38.

In FIG. 3B an embodiment comprising even more electrodes is illustrated. Two electrodes 39, 300 are generating an AC current through a section comprising three sets of measuring electrodes. This embodiment allows for measuring and comparing axial forces applied along segments of the probe. The embodiment further includes a balloon 301 attached to the segment between the electrodes and the distal end. The balloon may be provided in order to immobilise of the probe inside an object. The probe may also be immobilised in a proximal end e.g. by clamp connection to the nose, mouth etc. The probe may be provided with tubing with one or more openings 303 for filling and emptying the balloon with a pressurising fluid, thereby to provide a fluid connection between the inside of the balloon and an externally accessible opening 37. The probe may further be provided with means 302 for measuring the pressure inside channel. Combination with e.g. pressure measurement in the chamber may be advantageous in the determination of tissue forces and deformation in various directions and in cases where the perfusion has a function in keeping the conductor channel open in very elastic probes. Pressures may also be measured inside the balloon and anywhere along the probe.

In FIG. 3C an even further embodiment is illustrated. Here the balloon is provided with means 305 for measuring a physical quantity inside the balloon, such as the average cross-sectional area of the balloon by means of impedance planimetry or imaging technology, pressure applied to the balloon, etc. Additional equipment may be provided to the probe, e.g. inside the balloon. For example, an ultrasound transducer may be provided in order to monitor a wall change of the system surrounding the probe, such as to determine the stress of the surrounding wall tissue. Thereby a correlation between a wall change of the system surroundings of the probe and a deformation of the probe may be provided.

For the electrodes shown in FIG. 3, no connecting wires are shown, it is however to be understood that such wires, or alternative means, are present, e.g. in a separate cavity or lumen inside the catheter, or in any suitable way for providing electrical access between the electrodes and an exterior wire. The externally accessible wires are illustrated by reference numeral 38, as an alternative the system may be wireless. Also different channel ends 35-37 are illustrated. These channels may be used for providing fluid into the probe for various purposes, as mentioned above. The exterior tubing, wires etc. may be connected to various control and/or measuring equipment, inclusive electronic equipment.

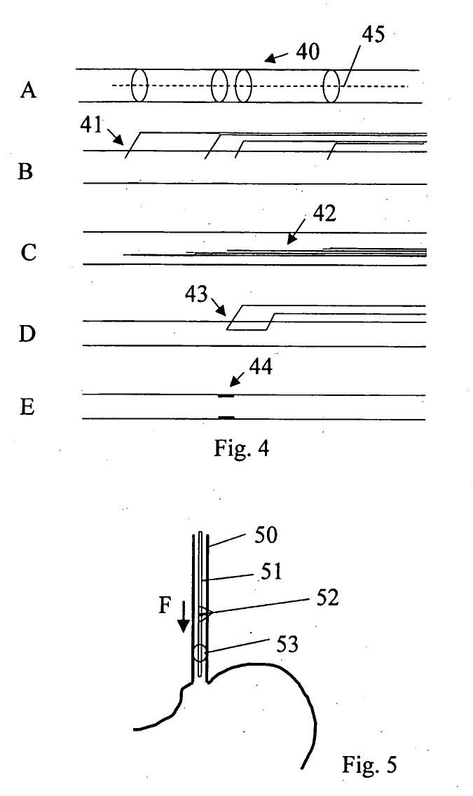

As illustrated in FIG. 4, different configurations of the electrodes may be envisioned. In FIG. 4A ring electrodes 40 placed inside a fluid filled channel are illustrated. In FIG. 4B the electrodes 41 are wires poked through the channel wall, where only the part of the wires inside the channel is without insulation material. In FIG. 4C the electrodes 42 are wires with a tip probe termination which are placed freely inside the lumen of the conducting medium, but with a fixed distance between the un-insulated tips of the wires. In FIG. 4D a stretchable wire 43 or medium is placed inside the probe, and in FIG. 4E two electrodes 44 are placed at the same circumferential level for measuring of deformation of the probe in the radial direction (wires not shown).

Electrodes are normally placed along the elongation of the probe, i.e. along an axis extending along the elongation of the elongated probe (the probe being stretched or compressed), however electrodes may also be placed in order to provide information about forces and deformations in other directions than in the longitudinal direction such as circumferentially or transversely to the length of the system. In general, the electrodes may be spaced along a reference curve 45 of the probe. A reference curve may be an imaginary curve extending along the probe, e.g. a reference curve extend along an outer surface or a centre axis of the probe, or extending along a circumference of the probe, spiralling along an outer surface of the probe, etc. The detection in multiple directions provides information about more complicated deformations of the system e.g. bending, twisting, shearing or the like.

The conducting medium may also be a solid of some substance capable of conducting an electrical current between the electrodes. If the conducting medium is a solid, the conductor may be introduced into a channel of the probe. Alternatively, the conductor may be introduced during manufacturing of the probe so that the conductor constitutes part of the probe itself. A solid medium may be such a medium as a soft metal, a polymer, a ceramics, a composite and/or natural materials. The medium may exhibit piezo-resistive and/or piezo electric properties.

In case the probe is to be introduced into a bodily system of a human or an animal, perhaps introduced into a bodily hollow system of the person or the animal, the choice of conducting medium may also be chosen as a substance or a material being non-harmful to the human or animal body. Such a criteria will be fulfilled by an acid like HCl in the stomach, or such as bile salts in the small intestine, or such as water with a suitable solution of NaCl in the esophagus.

Other materials than PVC may be chosen as the material which the probe is made of. PVC and the dimensions of the probe compared to the force being applied in the diagram shown will only result in a small elastic stretching of the probe. This may be beneficial in systems where it is important that the probe is fixed in relation to the deformations of the system. Other materials and other dimensions of the probe exhibiting more profound deformations, when a certain force is applied to the probe, may be suitable in systems, where it is important that the probe itself do not impede the deformations of the system. Depending on the mechanical properties of the probe itself, it may be necessary to correct for the material properties of the probe before a force or deformation of the bodily system can be determined with accuracy.

FIG. 5 provides an example of a use of the present invention. The probe 51 comprising electrodes 52 and a balloon 53 is inserted into the esophagus 50.

In the esophagus the probe may be immobilised by inflating the balloon. This causes the muscles surrounding the esophagus to try to drag the balloon and the probe away from the tract. The apparatus and method according to the invention provides a means for determining the reaction forces of the muscles of the esophagus in this situation, which may be used for scientific and/or diagnostic purposes, e.g. in order to determined the traction force during swallowing.

Although the present invention has been described in connection with preferred embodiments, it is not intended to be limited to the specific form set forth herein. Rather, the scope of the present invention is limited only by the accompanying claims.

In this section, certain specific details of the disclosed embodiment such as material choices, geometry of the apparatus or parts of the apparatus, techniques, measurement set-ups, etc., are set forth for purposes of explanation rather than limitation, so as to provide a clear and thorough understanding of the present invention. However, it should be understood readily by those skilled in this art, that the present invention may be practiced in other embodiments which do not conform exactly to the details set forth herein, without departing significantly from the spirit and scope of this disclosure. Further, in this context, and for the purposes of brevity and clarity, detailed descriptions of well-known apparatus, circuits and methodology have been omitted so as to avoid unnecessary detail and possible confusion.

1-35. (canceled)

36. An apparatus for measuring the deformation of a system, the apparatus comprising:
an elongated elastic probe,
a conducting medium attached to or contained by the probe, and
two or more electrodes being electrically connected by the conducting medium, the electrodes being attached to the probe,
wherein the apparatus furthermore comprising means for measuring an electrical parameter between at least two of the number of electrodes, the measured electrical parameter being indicative of a deformation of the probe in at least the longitudinal direction of the elongated probe.

37. An apparatus according to claim 36 comprising four or more electrodes, wherein at least two of the four or more electrodes are measuring electrodes comprising means for measuring the electrical potential between them, and wherein other at least two of the four or more electrodes are generating electrodes comprising means for generating an AC-field between the measuring electrodes.

38. An apparatus according to claim 36, wherein the measured electrical parameter is indicative of a force of a certain magnitude being applied to the probe.

39. An apparatus according to claim 38, wherein the force or the deformation are deduced from the measured electrical parameter by means of a pre-determined calibration function.

40. An apparatus according to claim 36, wherein the apparatus further comprising timer means for determining a timing of a change of the measured electrical parameter.

41. An apparatus according to claim 36, wherein the more than two electrodes are placed along a reference curve of the probe.

42. An apparatus according to claim 41, where the reference curve is a longitudinal axis extending along the elongation of the elongate probe, and thereby the more than two electrodes are placed along the longitudinal axis.

43. An apparatus according to claim 36, wherein the conducting medium is a liquid medium serving as an electrolyte for conducting the electric current between the electrodes.

44. An apparatus according to claim 43, wherein the liquid medium is a liquid preferably non-harmful to the bodily hollow system or the engineered structure being stimulated, such as an acid like HCl in the stomach, or such as bile salts in the small intestine, or such as water with NaCl in the esophagus.

45. An apparatus according to claim 36, wherein the conducting medium is a solid medium, such as compounds including at least one substance selected from the group of: soft metals, polymers, ceramics, composites and natural materials.

46. An apparatus according to claim 36, further comprising at least one inflatable balloon situated between a proximal end and a distal end of the probe, and the apparatus comprising means for passing an inflating fluid, preferably a liquid, from the proximal end to the balloon, and where the apparatus optionally is provided with means for measuring at least one physical properties of the balloon.

47. An apparatus according to claim 36, further comprising means for passing a chemical substance through one or more channels inside the probe to a number of openings in side-walls of the probe and out into the hollow system.

48. An apparatus according to claim 36, further comprising means for passing an electrical current through a number of wires in a number of the canals inside the probe, and when passing the electrical current to an outer surface of the probe, the outer surface being a surface abutting the inner wall of the hollow system.

49. A method for measuring a deformation of a system by introducing into the system an elongated elastic probe, the probe comprising:
a conducting medium attached to or contained by the probe, and
two or more electrodes being electrically connected by the conducting medium, the electrodes being attached to the probe,
wherein a deformation being indicative of a deformation of the probe in at least the longitudinal direction of the elongated probe is measured by measuring an electrical parameter between at least two of the two or more electrodes.

50. A method according to claim 49, comprising four or more electrodes, wherein at least two of the four or more electrodes are generating electrodes generating an AC-field between at least two other electrodes being measuring electrodes, the measuring electrodes measuring the electrical potential between them.

51. A method according to claim 49, wherein the deformation of the probe is indicative of a force of a certain magnitude being applied to the probe.

52. A method according to claim 51, wherein the force or the change in distance are deduced from the measured electrical parameter by means of a pre-determined calibration function.

53. A method according to claim 49, wherein a timing of a change of the measured electrical parameter is being determined, so as to obtain a measurement of a velocity and/or an acceleration of the deformation of the probe.

54. A method according to claim 49, wherein the more than two electrodes are placed along a reference curve of the probe, and wherein the physical quantities, such as the forces, the distances, the acceleration or the speed deduced from at least the measured electrical parameter between at least two electrodes, are quantities measured along a direction defined by the reference curve.

55. A method according to claim 54, wherein the reference curve is a longitudinal axis extending along the elongate probe, thereby the physical quantities measured between the at least two electrodes are quantities along a substantial longitudinal extension of the probe.

56. A method according to claim 49, wherein the probe is being provided with at least one inflatable balloon situated between a proximal end and a distal end of the probe, and where the method comprises the further step of inflating the at least one balloon, until the balloon abuts an inner wall of the system in order for the balloon and the probe to be fixed longitudinally in relation to the system.

57. A method according to claim 56, wherein the measuring of the electrical parameter between at least two of the two or more electrodes is obtain in correlation with a pressure change inside the balloon, a volume change of the balloon, a determination of the cross-sectional area of the balloon or other changes of the balloon.

58. A method according to claim 56, wherein the measuring of the electrical parameter between at least two of the two or more electrodes is obtain in correlation with a wall change of the system surroundings of the probe

59. A method according to claim 49, wherein a measurement during thermal stimulus is performed, when the probe is filled with a fluid, preferably a liquid, the liquid introducing a change in temperature of the probe and/or balloon and thus of an outer surface of the probe and/or balloon, the outer surface being a surface abutting the inner wall of the system, and where the deformation of the system is measured in correlation with the temperature of the fluid inside the probe and/or balloon.

60. A method according to claim 49, wherein a measurement during chemical stimulus is performed, when passing of a chemical substance through a number of the canals inside the probe to a number of openings in side-walls of the probe and out into the hollow system, and where the extension or the contraction of the hollow system is measured in correlation with the composition of the chemical substance.

61. A method according to claim 60, wherein the method is performed for measuring the passage of the chemical substance past a part of the probe abutting the internal wall of the system, the passage being indicative of the ability of the system to exercise a restraining influence, alternatively to exercise a passing influence, on liquids and solids.

62. A method according to claim 49, wherein a measurement during an electrical stimulus is performed, when passing an electrical current through a number of wires in a number of the canals inside the probe, and when passing the electrical current to an outer surface of the probe, the outer surface being a surface abutting the inner wall of the hollow system, and where the extension or the contraction of the hollow system is measured in correlation with the magnitude of the electrical current applied.

63. A method according to claim 49, wherein the method is performed anywhere in one of the following bodily systems: the muscles, the connective tissue, the skin, the bones, or where the method is performed anywhere in one of the following bodily hollow systems: the digestive system including the stomach, the urogenital tract including the bladder, the cardiovascular system including the heart, the lymph system, the ear canal including the eustachian canal and the posterior nares.

64. Apparatus according to claim 36, for use of subjecting a number of artificially applied stimuli to a bodily hollow system of a person or an animal, the stimuli being any of the stimuli: mechanical stimulus, thermal stimulus, chemical stimulus and electric stimulus.

65. Apparatus according to claim 36 for performing measurements in part of the digestive system including the stomach, preferably performing measurements in the gastrointestinal tract based on a prior stimulation of any of the following kinds: mechanical stimulus, thermal stimulus, chemical stimulus and electric stimulus.

66. Apparatus according to claim 36 for performing measurements in part of the urogenital system of a person or an animal, the urogenital system including the urinary bladder based on a prior stimulation of any of the following kinds: mechanical stimulus, thermal stimulus, chemical stimulus and electric stimulus.

67. Apparatus according to claim 49 for performing measurements in part of the cardiovascular system of a person or an animal, the cardiovascular system including the heart and the lymph system, based on a prior stimulation of any of the following kinds: mechanical stimulus, thermal stimulus, chemical stimulus and electric stimulus.

68. Apparatus according to claim 36 for performing measurements in part of the tissue of a person or an animal, the tissue including epitheliuous tissue, connective tissue, skin, and adipose tissue, based on a prior stimulation of any of the following kinds: mechanical stimulus, thermal stimulus, chemical stimulus and electric stimulus.

69. Apparatus according to claim 36 for performing measurements in part of the motoric system of a person or an animal, the motoric system including the muscles and the bones, based on a prior stimulation of any of the following kinds: mechanical stimulus, thermal stimulus, chemical stimulus and electric stimulus.

70. Apparatus according to claim 36 for performing measurement in non-human and non-animal systems such as in plants and in engineered structures.
